(19) United States
(12) Patent Application Publication
(10) Pub. No.: US 2004/0219143 A1
(43) Pub. Date: Nov. 4, 2004

(54) Methods for recombinant immunoglobulin treatment

(76) Inventor: Martin Heath Bluth, Brooklyn, NY (US)

Correspondence Address: Ryan, Mason & Lewis, LLP, 90 Forest Avenue, Locust Valley, NY, 11560, US

(21) Appl. No.: 10/426,334

(22) Filed: Apr. 30, 2003

(51) Int. Cl. A61K 39/395

(52) U.S. Cl. 424/131.1

(57) ABSTRACT

Techniques for immunotherapy are provided. The techniques provide: 1) a method for the treatment of a patient, comprising the steps of obtaining recombinant Fc fragments; and providing the recombinant Fc fragments for treatment of the patient; 2) a method for the treatment of a patient having an autoimmune disease, comprising the steps of obtaining recombinant Fc fragments of immunoglobulin G; and providing the recombinant Fc fragments of immunoglobulin G for treatment of the patient having the autoimmune disease; 3) a method for the treatment of a patient having an atopic disease, comprising the steps of obtaining Fc fragments of immunoglobulin G; and providing the Fc fragments of immunoglobulin G for treatment of the patient having the atopic disease; and 4) a method for the treatment of a human, comprising the steps of obtaining Fc fragments; and providing the Fc fragments for treatment of the human.

### FIELD OF THE INVENTION

The present invention relates to immunotherapy and, more particularly, to immunoglobulin treatment of a patient using recombinant immunoglobulin.

### BACKGROUND OF THE INVENTION

Immunoglobulin treatment, or treatment with antibodies, involves introducing exogenous antibodies to a patient. Immunoglobulin treatment has been used extensively for the treatment of multiple disease states in patients, including autoimmune diseases. Autoimmune diseases occur when the body's immune system attacks itself. Specifically, the body produces autoantibodies that cause the attack of body tissues. Immunoglobulin treatment functions to decrease the effects of the autoantibodies. First, immunoglobulin treatment introduces exogenous antibodies to the patient, thus diluting the effects of the autoantibodies by competing with them. Second, the introduction of exogenous antibodies is thought to stimulate the catabolism of autoantibodies in the patient.

Immunoglobulin treatment typically involves the use of concentrated antibodies extracted from pooled human donor serum. The pooled human donor serum may then be fractionated to isolate the antibodies present therein. Pooled human donor serum contains multiple classes of immunoglobulin (or antibody) molecules, typically only a fraction of which are needed for treatment. While it is possible to quantify an amount of each class of molecule in the pooled human donor serum, it is impractical to quantify each individual preparation. Thus, clinicians typically administer a preparation based on generic parameters such as a patient's body weight.

Further, immunoglobulin treatments may require that only a certain class or classes of molecule be given. However, with an immunoglobulin preparation derived from human donor serum, other classes of immunoglobulin are likely present. The presence of the other classes of immunoglobulin not needed for treatment may be especially problematic if the patient receiving treatment requires one class of immunoglobulin and has an adverse reaction to the other classes present in the preparation.

Additionally, although precautions are taken to ensure that the immunoglobulin preparation derived from human donor serum is free of contagious blood-born diseases and pathogens, such as human immunodeficiency virus (HIV) or hepatitis C, the presence of these factors remains a concern, and virus transmission remains a risk.

Conventional immunoglobulin treatments cause some notable side effects. For example, the treatments might result in hypersensitivity in the patient. As such, attempts have been made to eliminate the portions of the immunoglobulin molecule causing such side effects. In S. Lin, et al., “Giving Inhibitory Receptors a Boost,” Science, v. 291, p. 445-46 (2001), for example, administering the Fc_{γ} portion of the immunoglobulin molecule was found to be an effective treatment for immune thrombocytopenia. However, the studies were directed solely to the mouse model. Nonetheless, contagion transmission remains a concern.

Thus, there exists a need for an immunoglobulin treatment that includes a quantifiable amount of immunoglobulin, does not bring about the harmful side effects commonly associated with immunoglobulin therapy and avoids the risks associated with human derived donor serum.

### SUMMARY OF THE INVENTION

The present invention provides techniques for immunotherapy. In one aspect of the invention, the technique provides a method for the treatment of a patient. The method comprises the steps of: obtaining recombinant Fc fragments; and providing the recombinant Fc fragments for treatment of the patient. The recombinant immunoglobulin may be administered to a patient, e.g., intravenously, intramuscularly, subcutaneously, or as an inhalant.

In another aspect of the invention, the technique provides a method for the treatment of a patient having an autoimmune disease. The method comprises the steps of: obtaining recombinant Fc fragments of immunoglobulin G; and providing the recombinant Fc fragments of immunoglobulin G for treatment of the patient having an autoimmune disease.

In a further aspect of the invention, the technique provides a method for the treatment of a patient having an atopic disease, such as asthma. The method comprises the steps of: obtaining Fc fragments of immunoglobulin G; and providing the Fc fragments of immunoglobulin G for treatment of the patient having the atopic disease.

In yet another aspect of the invention, the technique provides a method for the treatment of a human. The method comprises the steps of: obtaining Fc fragments; and providing the Fc fragments for treatment of the human.

A more complete understanding of the present invention, as well as further features and advantages of the present invention, will be obtained by reference to the following detailed description and drawings.

### BRIEF DESCRIPTION OF THE DRAWINGS

FIG. 1 is a diagram illustrating a tertiary structure of an immunoglobulin G (IgG) molecule; and

FIG. 2 is a flow chart illustrating an exemplary methodology for treatment according to an embodiment of the present invention.

### DETAILED DESCRIPTION OF PREFERRED EMBODIMENTS

The present invention will be described in the context of an illustrative treatment using Fc fragments of immunoglobulin G (IgG). The Fc fragments may be recombinant. However, it is to be understood that the teachings presented herein are more generally applicable to treatments using immunoglobulin molecules and should not be construed as being limited to any particular immunoglobulin molecule, part or form thereof.

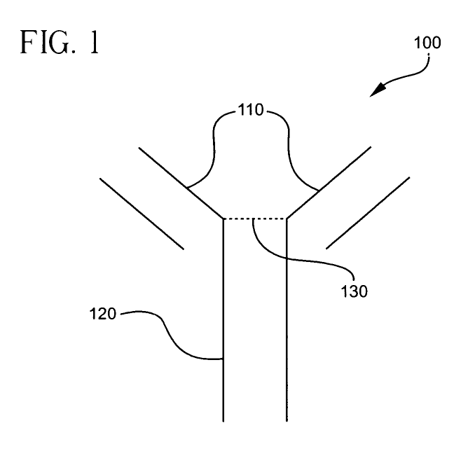

FIG. 1 is a diagram illustrating a tertiary structure of an IgG molecule 100. There are five identified classes of immunoglobulin molecules, including immunoglobulin A (IgA), immunoglobulin D (IgD), immunoglobulin E (IgE), immunoglobulin G (IgG) and immunoglobulin M (IgM). Immunoglobulin molecules, i.e., antibodies, are produced by the body as part of an immune response. The class, or classes, of immunoglobulin molecules produced depends on the stage of, or type of, immune response. For example, predominately IgG molecules are encountered in the blood and lymph during secondary response reactions. IgE molecules bind to specific sites on mast cells and thus play an important role in allergic reactions. IgA is predominately secreted in the respiratory, digestive and urogenital tracts.

As shown in FIG. 1, IgG molecule 100 comprises four polypeptide chains arranged into distinct regions, or fragments. The fragments comprise antigen-binding fragments (Fab fragments) 110 and constant fragment (Fc fragment) 120. Thus, as used herein, the term ‘fragment’ will be used to describe fragments of IgG molecule 100, i.e., Fab fragments 110 and Fc fragment 120, as compared to intact IgG molecule 100. Each of Fab fragments 110 comprises a combining site that is specific for combining to a portion, i.e., an epitope, of a particular antigen, or antigens. Because the body encounters a plethora of antigens, and since the combining site of each of Fab fragments 110 is specific for a small number of those antigens, Fab fragments 110 comprise variable regions to ensure a greater diversity of antibody specificity. As such, a given immunoglobulin molecule comprises variations, as compared to other immunoglobulin molecules, making the given immunoglobulin molecule specific for combining with a distinct epitope.

Fab fragments 110 are connected to Fc fragment 120 by hinge region 130. Hinge region 130 comprises a disulfide bond. Disulfide bonds occur throughout IgG molecule 100 to bind the polypeptide chains. Fc fragment 120 functions in binding IgG molecule 100 to a cell surface. For example, following an encounter with an antigen, plasma cells within the body may secrete immunoglobulin molecules specific for epitopes of that antigen. The immunoglobulin molecules secreted may then be displayed on the surfaces of B-cells. B-cells, or B-lymphocytes, mediate humoral immunity. The immunoglobulin molecule is attached to the B-cell surface by Fc fragment 120.

Additionally, the surfaces of the B-cells are generally less variable than the epitopes of antigens. Thus, Fc fragment 120 comprises constant regions, regions that remain relatively invariant from one immunoglobulin molecule to another of the same class, i.e., heavy chain isotype, as described below.

Each IgG molecule comprises two types of polypeptide chains, heavy chains and light chains. The terms “heavy” and “light” refer to the molecular masses of the chains. Heavy chains have a molecular mass of about 50,000 to about 70,000 daltons. Light chains have a molecular mass of about 23,000 daltons. Within each of the classes of immunoglobulin molecules, namely, IgA, IgD, IgE, IgG and IgM, there are only two types of polypeptide light chains, kappa (κ) and lambda (λ). However, each of the five classes of immunoglobulin molecules comprises a unique type of heavy chain. There are thus five types of heavy chains, α, δ, ε, γ and μ, that correspond to IgA, IgD, IgE, IgG and IgM, respectively.

The Fc fragment of each immunoglobulin molecule comprises predominately heavy chain polypeptides. Thus, when addressing the Fc fragment of a particular class of immunoglobulin molecule, it is common practice to refer to the heavy chain designation for that class. For example, the Fc fragment of IgG may be referred to as Fc_{γ}. As such, hereinafter, the Fc fragment of IgG will be referred to as Fc_{γ}.

Further, there exists a number of heavy chain subclasses for certain classes of immunoglobulin molecule. Four heavy chain subclasses of IgG exist, i.e., IgG1, IgG2, IgG3 and IgG4, and two heavy chain subclasses of IgA exist, i.e., IgA1 and IgA2. Further, it is to be understood that any reference herein to Fc_{γ} applies generally to the Fc_{γ} of all the heavy chain subclasses of IgG.

A particular heavy chain subclass may be implicated in certain diseases. For instance, IgG1 and IgG3 play a role in hemolytic diseases of the newborn (HDN). For a detailed description of the effects of IgG1 and IgG3 in HDN, see P. Lambin, et al., “IgG1 and IgG3 anti-D In Maternal Serum and On the RBCs of Infants Suffering From HDN: Relationship With the Severity of the Disease,” Transfusion, v. 42, p. 1537-46 (2002), the disclosure of which is incorporated by reference herein.

The polypeptide sequence and corresponding genetic, i.e., deoxyribonucleic acid (DNA), sequence of Fc_{γ} have been identified by researchers and are publicly available. For example, the polypeptide and genetic sequences are available in GenBank, a public database maintained by the National Center for Biotechnology Information (NCBI). A new release of GenBank is made every two months. The GenBank entry for the Fc_{γ} of the IgG1 heavy chain subclass is as follows:

```
LOCUS AF237583 1827 bp DNA linear PRI
11-MAY-2001
DEFINITION Homo sapiens recombinant IgG1 heavy chain gene, partial
cds.
ACCESSION AF237583
VERSION AF237583.1 GI:9857752
KEYWORDS
SOURCE Homo sapiens (human)
  ORGANISM Homo sapiens
      Eukaryota; Metazoa; Chordata; Craniata; Vertebrata;
Euteleostomi;
      Mammalia; Eutheria; Primates; Catarrhini; Hominidae; Homo.
REFERENCE 1 (bases 1 to 1827)
  AUTHORS Vidarsson, G., van der Pol, W. L., van den Elsen, J. M. H.,
Vile, H.,
      Jansen, M., Duijs, J., Morton, H. C., Boel, E., Daha, M. R.,
Corthesy, B.
      and van de Winkel, J. G. J.
  TITLE Activity of human IgG and IgA subclasses in immune
defense against
      Neisseria meningitidis serogroup B
  JOURNAL J. Immunol. 166 (10), 6250-6256 (2001)
  MEDLINE 21240692
  PUBMED 11342648
REFERENCE 2 (bases 1 to 1827)
```

| | |
|---|---|
| AUTHORS | Vidarsson, G., Jansen, M., Boel, E. and van de Winkel, |
| | J. G. J. |
| TITLE | Direct Submission |
| JOURNAL | Submitted (22-FEB-2000) Department of Immunology, |
| University | |

```
      Medical Center Utrecht, Rm. KC.02-085.2, Lundlaan 6,
Utrecht 3584
      EA, The Netherlands
```

| | |
|---|---|
| FEATURES | Location/Qualifiers |

| | |
|---|---|
| source | 1 . . . 1827 |

/organism = “Homo sapiens”
/db_xref = “taxon:9606”

| | |
|---|---|
| mRNA | join(<1 . . . 294, 686 . . . 730, 849 . . . 1178, |
| | 1276 . . . >1598) |

/product = “recombinant IgG1 heavy chain”

| | |
|---|---|
| CDS | join(<1 . . . 294, 686 . . . 730, 849 . . . 1178, |
| | 1276 . . . 1598) |

| | /codon_start = 3 |
|---|---|
| | /product = “recombinant IgG1 heavy chain” |
| | /protein_id = “AAG00909.1” |
| | /db_xref = “GI:9857753” |
| | /translation = “SEQ ID NO: 1” |
| exon | 1 . . . 294 |
| | /note = “CH1” |
| exon | 686 . . . 730 |
| | /note = “hinge” |
| exon | 849 . . . 1178 |
| | /note = “CH2” |
| exon | 1276 . . . 1598 |
| | /note = “CH3” |

BASE COUNT 390 a 621 c 498 g 316 t 2 others
ORIGIN
{SEQ ID NO: 2}

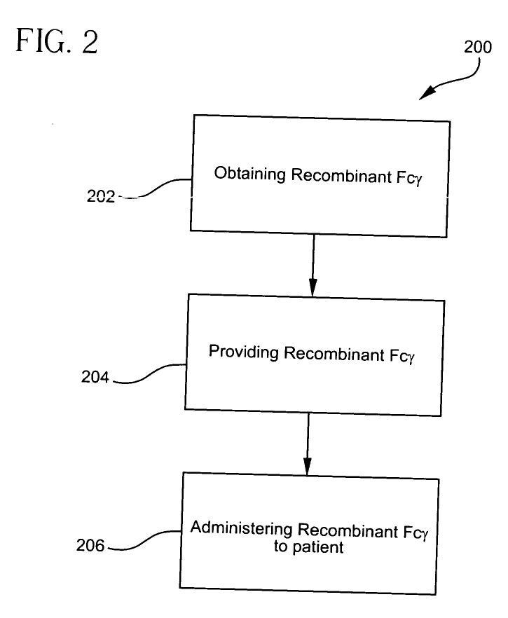

FIG. 2 is a flow chart illustrating an exemplary methodology 200 for treatment according to an embodiment of the present invention. As shown in step 202, Fc fragments of IgG are obtained. Fc fragments of IgG may be obtained either according to the preparation steps described below, or as a pre-prepared allotment. Thus, a manufacturer may obtain Fc fragments of IgG by preparing allotments, e.g., for sale, whereas a physician might obtain Fc fragments of IgG from a manufacturer directly, or through an intermediary, such as a wholesaler or pharmacy.

In an exemplary embodiment, only Fc_{γ} is used for treatment. IgG molecule 100 can be fragmented using proteolytic treatment. Proteolytic treatment involves the use of proteolytic enzymes which function in the breakdown of proteins. The proteolytic treatment of IgG molecule 100 results in two Fab fragments 110 and one Fc fragment 120. Administering only Fc_{γ} is thought to provide comparable, effective, results as compared to administering intact IgG molecules. Further, as is described below, the Fc_{γ} may be recombinant. Thus, according to the teachings of the present invention, recombinant Fc_{γ} is prepared.

Treatments comprising Fab fragments 110 have been shown to cause detrimental effects. For example, it has been found that treatments comprising immunoglobulin light chains illicit immune hypersensitivity. As described above, only Fab fragments 110 comprise light chains (Fc fragment 120 comprises predominately heavy chains). Thus, treatments comprising Fab fragments 110 may cause illicit immune hypersensitivity. A detailed description of the hypersensitivity caused by immunoglobulin light chains may be found, for example, in M. Castro, “Immunoglobulin-Free Light Chains Elicit Immediate Hypersensitivity-Like Responses,” Nature Medicine, v. 8, no. 7, p. 694-701, the disclosure of which is incorporated by reference herein.

Treatments comprising intact immunoglobulin molecules, i.e., intact IgG molecule 100, also have the detrimental effect of binding complement. Complement is a series of blood plasma proteins that, acting as a part of the natural immune system, bind to extracellular pathogens, triggering their destruction. For example, when an invading microorganism is encountered, complement aids in destroying the microorganism. However, intact immunoglobulin molecules may crosslink. Crosslinked immunoglobulin molecules bind complement. As such, the crosslinked intact immunoglobulin molecules are ‘tagged’ as binding foreign matter and destroyed. Thus, treatments comprising intact immunoglobulin molecules lose effectiveness. A possible benefit of the treatments of the present invention comprising fragments of immunoglobulin molecules is that the fragments, i.e., Fc fragment 120, are not known to crosslink. Although the present invention discloses monomeric Fc fragments, it is to be understood that the teachings herein are further applicable to multimeric Fc fragments. As such, the fragments in the treatment may not bind complement, making the treatment more effective than treatments comprising intact immunoglobulin molecules.

The mass production of Fc fragments using current techniques involving proteolytic treatment requires that IgG molecule 100 be isolated from a pool of human donor serum. Fc_{γ} may be prepared using proteolytic treatment by first isolating intact IgG from pooled human donor serum. In general, isolating immunoglobulin molecules from pooled human donor serum may be performed by Cohn-Oncley cold ethanol fractionation, wherein the plasma is purified by precipitating protein fractions at varying levels of ethanol, salt and pH. Following the fractionation, ultrafiltration and ion exchange chromatography may be performed to further purify the sample. Once intact IgG is isolated, as described above, the IgG molecule may be fragmented resulting in two Fab fragments 110 and one Fc fragment 120. The Fc fragments, i.e., Fc_{γ}, may then be obtained using standard extraction methods. The use of donor IgG molecule 100, however, aside from being dependent on having a constant pool of donors, is costly, time consuming and brings about the risk of the patient contracting blood-born diseases and pathogens.

In contrast, recombinant Fc_{γ} may be mass-produced by a manufacturer, or alternatively, individual or small batch preparations may be prepared by researchers or clinicians. The term “clinician” refers to any person capable of administering treatment to a patient. An exemplary clinician includes, but is not limited to, a physician. Clinicians may further include health care workers, nurses, nurse aides, home health aides, physician assistants and the like.

Recombinant Fc_{γ} may be obtained using genetic engineering techniques, i.e., recombinant technology. Recombinant technology, used according to the teachings of the present invention, involves introducing the target genetic material, for example DNA, into a host organism, i.e., a slave cell, the target genetic material encoding the target polypeptide sequences comprising Fc_{γ}. A typical slave cell is Escherichia coli (E. coli). The slave cell will replicate the target genetic material along with the genetic material of the host organism. The host organism will translate the target genetic material into polypeptide sequences, i.e., the primary structure of Fc_{γ}. The Fc_{γ} fragments will be identical copies of the target Fc_{γ} fragments.

Other suitable slave cells include yeast, mammalian cells, e.g., Chinese hamster ovary (CHO) cells, as well as other applicable malignant cell lines. A potential benefit of using CHO cells as the slave cell is that CHO cells, as with other eukaryotic cells, have the ability to glycosylate proteins. Thus, in an embodiment of the present invention, CHO slave cells are employed to yield Fc_{γ} that may be glycosylated.

An exemplary technique for producing recombinant Fc_{γ} is described in L. Jendeberg et al., “Engineering of Fc₁ and Fc₃ from Human Immunoglobulin G to Analyze Subclass Specificity for Staphylococcal Protein A,” Journal of Imin. Methods, v. 201, p. 25-34 (1997), the disclosure of which is incorporated by reference herein. The technique was developed for structural and functional studies of the Fc region of an intact immunoglobulin molecule. The technique provides that two different strains of E. coli be used as hosts, one for replicating the genetic material and another for translating the genetic material into proteins. E. coli strain RR1ΔM15 was used for replicating the genetic material. E. coli strain KS476 was used for translating the genetic material into proteins.

The genetic material was isolated from human spleen cells. The isolated genetic material was amplified using polymerase chain reaction (PCR) carried out using a Techne PHC-I Thermocycler®. PCR allows for the production of multiple copies of a sample of genetic material. Thus, researchers, when working with a small or limited sample of genetic material, will use PCR to generate enough genetic material for use or experimentation.

The overall sequences of genetic material generated by PCR were digested using the endonucleases EcoRI and HindIII, resulting in smaller fragments. Additionally, the restriction endonuclease NsiI was used to cleave the genetic material from the E. coli host.

The specific technique, including the host organism, the PCR technique and apparatus and the endonucleases described herein are provided as a typical exemplary methodology for producing recombinant Fc_{γ}, and may be used in accordance with the teachings of the present invention. However, the teachings of the present invention should not be construed as being limited to any particular methodology for producing recombinant Fc_{γ}.

The use of recombinant technology to generate recombinant Fc_{γ} fragments has important beneficial properties. First, the use of recombinant technology subverts the possibility of transmitting blood-born diseases or pathogens. Recombinant technology allows researchers and clinicians to isolate the desired Fc_{γ} fragment, according to the recombinant techniques described above, and then generate a plurality of copies. The use of pooled serum is thus avoided.

Second, the use of recombinant technology allows researchers to make preparations that include only the classes of immunoglobulin molecules, or parts thereof, that are needed for treatment. The ability to selectively include only certain classes of immunoglobulin molecules is beneficial when the patient receiving the treatment has a negative reaction to certain classes of immunoglobulin molecules, but not others. For example, a patient may have an adverse allergic reaction to treatment with IgA. Thus, according to the teachings of the present invention, the patient can receive treatment with a preparation in which IgA is selectively absent. This selectivity cannot be obtained with isolated fractions because pooled donor serum will likely contain a plurality of classes of immunoglobulin molecules, in varying amounts.

Third, the use of recombinant technology allows researchers to produce a quantifiable preparation of Fc_{γ}. As described above, researchers can prepare selective batches of recombinant Fc_{γ}. As such, researchers may also control the amount of recombinant Fc_{γ} in each batch. The ability to control batch amounts allows for careful monitoring and control of treatment. In contrast, treatment with isolated donor serum does not allow for such control. Donor serum will likely contain a predictable amount of each class of immunoglobulin molecule, however, it is not practically possible to quantify that amount for each treatment. Thus, physicians administering the treatment may only have an estimate of the quantity of each immunoglobulin heavy chain isotype.

The recombinant Fc_{γ} obtained may then be prepared as part of a solution, the solution to be administered to a patient as described below. The solution of recombinant Fc_{γ} may comprise less than or equal to about 95 weight percent (wt. %) recombinant Fc_{γ}, based on the total weight of the solution. Further, the solution of recombinant Fc_{γ} may comprise between about 1 to about 50 wt. % recombinant Fc_{γ}, based on the total weight of the solution.

As shown in step 204, the obtained recombinant Fc_{γ} is then provided for treatment of a patient. For example, the manufacturer might provide the obtained recombinant Fc_{γ} to a clinician for administering to a patient, further shown in step 206. While the present method described herein is presented in discrete steps and the description highlights different entities performing different steps, it is to be understood that according to the teachings herein, each of the steps may be performed independently, or concurrently, and in any combination. Further, it is to be understood that any of the steps, independently or in combination, may be performed by a single entity or any combination of entities. By way of example only, in an alternative embodiment, a clinician prepares recombinant Fc_{γ} and provides the recombinant Fc_{γ} for treatment of a patient. The patient may obtain the recombinant Fc_{γ} directly from the clinician and self-administer the recombinant Fc_{γ} treatment.

As shown in step 206, treatment may involve administering the recombinant Fc_{γ} to the patient. Immunoglobulin treatments may be administered using transfusion therapy. One type of transfusion therapy, intravenous immune globulin (IVIG) therapy, involves administering solutions intravenously. To be administered intravenously, the solutions predominately comprise small molecular weight complexes. Accordingly, the treatment of the present invention may be administered following the same methodologies as UVIG therapy. Thus, in an exemplary embodiment, Fc_{γ} is administered intravenously.

Another type of transfusion therapy, intramuscular immune globulin (IG) therapy, involves administering solutions intramuscularly. The solutions may comprise high molecular complexes. While solutions comprising high molecular weight complexes may be suitable for IG therapy, the use of such solutions in IVIG therapy would be dangerous. Accordingly, the treatment of the present invention may be administered following the same methodologies as IG therapy. Thus, in an exemplary embodiment, Fc_{γ} is administered intramuscularly.

While treatment dosage may be standardized according to easily ascertainable patient characteristics, such as body weight or age, other patient characteristics may be factored into determining proper dosing. The other characteristics include, but are not limited to, the severity of the condition being treated, the vital statistics of the patient, and the like. Typically, treatment begins by administering doses less than the determined optimum dosage. The dosages may be increased incrementally until the desired treatment affect is achieved. In an exemplary embodiment wherein treatment is administered intravenously or intramuscularly, dosage is determined based on the body weight of the patient. The treatment may be administered to the patient in a dosage amount of between about one picogram (pg) per kilogram (kg) per day (d) (pg/kg/d) to about three grams (g) per kg per d (g/kg/d), wherein the kg value represents the weight of the patient. Further, the solution may be administered to the patient in a dosage amount of between about 75 micrograms (μg) per kg per d (μg/kg/d) to about 400 milligrams (mg) per kg per d (mg/kg/d). Treatment may be administered for up to about seven days, although the treatment time may vary depending on factors such as the dosage and the condition of the patient. Treatment may be repeated every about one to about six months from the initial treatment.

For treatments being administered intravenously or intramuscularly, the solutions must be prepared in a suitable, injectable and sterile, form. Suitable injectable forms include, but are not limited to, aqueous solutions and dispersions prepared in carriers such as water, ethanol, glycerol, propylene glycol, liquid polyethylene glycol, vegetable oils, and the like. Further, the solutions should be prepared and stored in a sterile form and be adequately protected against contamination by microorganisms, such as fungi, bacteria and viruses. Contamination may be prevented by the use of antimicrobial agents such as parabens, chlorobutanol, phenol, sorbic acid, thimerosal, and the like.

In another exemplary embodiment, Fc_{γ} is administered to the patient as an inhalant. The inhalant may be in the form of an aerosol. Fc_{γ} administered as an inhalant allows for the direct treatment of areas of the respiratory tract. Thus, administering Fc_{γ} in the form of an inhalant is useful for, but not limited to, the treatment of respiratory disorders or diseases, for example, asthma and asthma-related conditions.

In the embodiment wherein Fc_{γ} is administered as an inhalant, the Fc_{γ} should be contained in, or formed into, particles of a size sufficiently small to pass through the mouth and larynx upon inhalation and into the bronchi and alveoli of the lungs. The particles should have a size in the range of about one to about ten microns in diameter.

In a further exemplary embodiment, Fc_{γ} is administered to the patient topically. Topical applications are particularly useful for direct localized treatment. Topical applications may include the application of topical treatments, including but not limited to, ointments, creams, transdermal patches, as well as any combination of the foregoing topical treatments. Ointments or creams may be prepared comprising Fc_{γ} and a suitable ointment or cream delivery medium. The ointment or cream may be applied to the areas of the patient requiring the treatment. The Fc_{γ} contained in the ointment or cream will diffuse transdermally into the body of the patient providing treatment to the effected area.

Additionally, as mentioned above, Fc_{γ} may be administered using a transdermal patch. The transdermal patch may be worn on the skin of the patient like a bandage. The transdermal patch allows for a prolonged treatment to be administered. For example, the patient may wear the transdermal patch for a plurality of hours and receive low dose treatments throughout that period. Other applicable treatment methods may be used in accordance with the teachings of the present invention. For example, a solution comprising Fc_{γ} may be injected subcutaneously.

The foregoing techniques are provided merely as exemplary methodologies for administering treatment to a patient and it is to be understood that the teachings of the present invention are generally applicable to any suitable methodology and should not be limited to any particular techniques described herein.

The foregoing techniques may be used to treat any disorder wherein the pathology lies in the Fc_{γ}-Fc_{γ} receptor (Fc_{γ}R) interaction. Further, in an exemplary embodiment, recombinant Fc_{γ} is used in the treatment of an autoimmune disease. The teachings of the present invention are applicable to the treatment of autoimmune diseases, including but not limited to, the following disease states: Guillain-Barre syndrome, Kawasaki syndrome, dermatomyositis, immune thrombocytopenic purpura (ITP), chronic inflammatory demylinating polyneuropathy, multifocal motor neuropathy, autoimmune hemolytic anemia, myasthenia gravis, Lambert-Eaton syndrome, Churg-Strauss vasculitides, multiple sclerosis, bullous pemphigoid, heparin-induced thrombocytopenia (HIT), post transfusion purpura (PTP), as well as any combination of the foregoing disease states.

In another exemplary embodiment, the Fc_{γ} is administered for the treatment of atopy. The Fc_{γ} administered may be recombinant Fc_{γ}. Atopy is the predisposition for developing an IgE-mediated response to common environmental allergens, i.e., atopic diseases such as hay fever, atopic dermatitis (eczema) and Job's syndrome. Further, atopy is the strongest identifiable predisposing factor for developing asthma.

Asthma is a disease that affects the airways of a patient, thus, making breathing difficult. During an asthma “attack” the muscles around the airways tighten and, thus, restrict the air moving in and out of the lungs. This condition causes the patient to find it difficult to breath. The symptoms of asthma include coughing, wheezing, shortness of breath and a tight feeling in the chest.

Recent research has determined that immunoglobulin treatment may be used in the treatment of asthma. Detailed descriptions of the research and the effects of immunoglobulin treatment on asthma are provided in D. Mimouni, et al., “Incidental Asthma Prevention by Immune Serum Globulin,” Ann Allergy Asthma Immunol v. 89, p. 99-100 (2002), the disclosure of which is incorporated by reference herein. Specifically, the treatments described herein are effective in decreasing IgE production. Thus, the recombinant Fc_{γ} of the present invention may be administered to treat atopic diseases, including asthma.

While the present invention has been described in accordance with the treatment of diseases and disorders described herein, it is to be understood that the teachings of the present invention are generally applicable to any diseases or disorders necessitating immunoglobulin treatment. Thus, the teachings of the present invention should not be construed as being limited to the treatment of any particular disease or disorder.

Although illustrative embodiments of the present invention have been described herein, it is to be understood that the invention is not limited to those precise embodiments, and that various other changes and modifications may be made by one skilled in the art without departing from the scope or spirit of the invention.

What is claimed is:

1. A method for treatment of a patient, the method comprising the steps of:
obtaining recombinant Fc fragments; and
providing the recombinant Fc fragments for treatment of the patient.

2. The method of claim 1, wherein at least one of the recombinant Fc fragments is a recombinant Fc fragment of immunoglobulin G.

3. The method of claim 2, wherein the recombinant Fc fragments are provided in a solution, the solution comprising less than or equal to about 95 weight percent recombinant Fc fragments of immunoglobulin G, based on the total weight of the solution.

4. The method of claim 1, wherein the patient has an autoimmune disease.

5. The method of claim 4, wherein the autoimmune disease comprises a disease state selected from the group consisting of Guillain-Barre syndrome, Kawasaki syndrome, dermatomyositis, immune thrombocytopenic purpura (ITP), chronic inflammatory demylinating polyneuropathy, multifocal motor neuropathy, autoimmune hemolytic anemia, myasthenia gravis, Lambert-Eaton syndrome, Churg-Strauss vasculitides, multiple sclerosis, bullous pemphigoid, heparin-induced thrombocytopenia (HIT), post transfusion purpura (PTP), and a combination comprising at least one of the foregoing disease states.

6. The method of claim 1, wherein the patient has an atopic disease.

7. The method of claim 6, wherein the atopic disease comprises a disease state selected from the group consisting of hay fever, atopic dermatitis, Job's syndrome, asthma, and a combination comprising at least one of the foregoing disease states.

8. The method of claim 6, wherein the atopic disease is asthma.

9. The method of claim 1, wherein the obtaining step further comprises the step of preparing the recombinant Fc fragments.

10. The method of claim 1, further comprising the step of administering the recombinant Fc fragments to the patient.

11. The method of claim 10, wherein the recombinant Fc fragments are administered intravenously.

12. The method of claim 10, wherein the recombinant Fc fragments are administered intramuscularly.

13. The method of claim 10, wherein the recombinant Fc fragments are administered as an inhalant.

14. The method of claim 13, wherein the inhalant comprises an aerosol.

15. The method of claim 10, wherein the recombinant Fc fragments are administered subcutaneously.

16. The method of claim 10, wherein the recombinant Fc fragments are administered topically.

17. The method of claim 16, wherein the topical administration comprises application of a topical treatment selected from the group consisting of ointments, creams, transdermal patches, and combinations comprising at least one of the foregoing topical treatments.

18. A method for treatment of a patient having an autoimmune disease, the method comprising the steps of:
obtaining recombinant Fc fragments of immunoglobulin G; and
providing the recombinant Fc fragments of immunoglobulin G for treatment of the patient having the autoimmune disease.

19. The method of claim 18, wherein the autoimmune disease comprises a disease state selected from the group consisting of Guillain-Barre syndrome, Kawasaki syndrome, dermatomyositis, immune thrombocytopenic purpura (ITP), chronic inflammatory demylinating polyneuropathy, multifocal motor neuropathy, autoimmune hemolytic anemia, myasthenia gravis, Lambert-Eaton syndrome, Churg-Strauss vasculitides, multiple sclerosis, bullous pemphigoid, heparin-induced thrombocytopenia (HIT), post transfusion purpura (PTP), and a combination comprising at least one of the foregoing disease states.

20. The method of claim 18, wherein the obtaining step further comprises the step of preparing the recombinant Fc fragments.

21. The method of claim 18, further comprising the step of administering the recombinant Fc fragments of immunoglobulin G to the patient having an autoimmune disease.

22. A method for treatment of a patient having an atopic disease, the method comprising the steps of:
obtaining Fc fragments of immunoglobulin G; and
providing the Fc fragments of immunoglobulin G for treatment of the patient having the atopic disease.

23. The method of claim 22, wherein at least one of the Fc fragments of immunoglobulin G is a recombinant Fc fragment of immunoglobulin G.

24. The method of claim 22, wherein the obtaining step further comprises the step of preparing the Fc fragments.

25. The method of claim 22, further comprising the step of administering the Fc fragments of immunoglobulin G to the patient having an atopic disease.

26. The method of claim 22, wherein the atopic disease comprises a disease state selected from the group consisting of hay fever, atopic dermatitis, Job's syndrome, asthma, and a combination comprising at least one of the foregoing disease states.

27. The method of claim 22, wherein the atopic disease is asthma.

28. A method for treatment of a human, the method comprising the steps of:
obtaining Fc fragments; and
providing the Fc fragments for treatment of the human.


SEQUENCE LISTING (2 sequences)

SEQ ID NO: 1 - protein, 329 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1827 bp, Homo sapiens
  misc_feature (1778)..(1778): n is a, c, g, or t
cctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcggtacccgggtggcatccctgtgacccctccccagtgcctctcctggccctggaagttgccactccagtgcccaccagccttgtcctaataaaattaagttgcatcattttgtctgactaggtggccttctataatattatggggtggaggggggtgggatggagcaaggggcaangttgggaagacaacctgtanggcctgcggggtctattgggaacccaact